(19) United States
(12) Patent Application Publication
(10) Pub. No.: US 2021/0106523 A1
(43) Pub. Date: Apr. 15, 2021

(54) IMMUNOSUPPRESSIVE DOSAGE FORMS AND METHODS OF USE

(71) Applicant: Gemini Laboratories, LLC, Bridgewater, NJ (US)

(72) Inventors: Padam Bansal, Bridgewater, NJ (US); Hardik Patel, Hillsborough, NJ (US); Amitkumar Patel, Edison, NJ (US); Suhaskumar Patel, Edison, NJ (US)

(73) Assignee: Gemini Laboratories, LLC, Bridgewater, NJ (US)

(21) Appl. No.: 16/982,084

(22) PCT Filed: Mar. 19, 2019
(86) PCT No.: PCT/US19/22989

§ 371 Date: Sep. 18, 2020

Related U.S. Application Data
(60) Provisional application No. 62/644,782, filed on Mar. 19, 2018.

(51) Int. Cl. A61K 9/00; A61K 31/436; A61K 47/26; A61K 47/24; A61K 47/02

(52) U.S. Cl. CPC A61K 9/0095; A61K 31/436; A61K 47/02; A61K 47/24; A61K 47/26

(57) ABSTRACT

The present disclosure relates to formulations, methods, kits, and dosage forms for oral pharmaceutical formulations comprising one or more immunosuppressive agents. The pharmaceutical formulations of the present disclosure can be useful in treating organ or stem cell transplant recipients or T-cell mediated diseases.

This application is the U.S. National Stage filing of International Patent Application Number PCT/US2019/022989, filed on Mar. 19, 2019, which claims the benefit of U.S. Provisional Patent Application Ser. No. 62/644,782 filed on Mar. 19, 2018, the contents of each are incorporated herein by reference in its entirety. This application also claims priority to U.S. patent application Ser. No. 16/813,813 filed Mar. 10, 2020, which claims the benefit of U.S. Provisional Patent Application Ser. No. 62/820,732, filed Mar. 19, 2019 the contents of each are incorporated herein by reference in its entirety.

### TECHNICAL FIELD

Embodiments of the present disclosure relate generally to formulations, methods, kits, and dosage forms of a pharmaceutical formulation that can be used for the treatment of organ and stem cell transplant recipients and for the treatment of other indications, including certain T-cell mediated diseases.

### BACKGROUND

Tacrolimus (also known as FK-506 or Fujimycin) is an immunosuppressive drug that can be used after organ or stem cell transplants to reduce the activity of the patient's immune system, and so lower the risk of organ rejection. It can reduce interleukin-2 (IL-2) production by T-cells. Tacrolimus can also be used in a topical preparation for the treatment of severe atopic dermatitis (eczema), severe refractory uveitis after bone marrow transplants, and the skin condition vitiligo. Tacrolimus is a 23-membered macrolide lactone discovered in 1987 from the fermentation broth of a Japanese soil sample that contained the bacteria Streptomyces tsukubaensis. The drug is sold under the trade names Prograf® given twice daily (oral) or as a continuous infusion (intravenous); Advagraf®, which is an extended release formulation allowing once daily dosing (oral); Envarsus,®, which is an extended release oral tablet with once daily dosing; and Protopic®, which is a topical formulation.

Tacrolimus has a poor solubility profile which presents challenges towards the development of a solution dosage form that could be administered to younger patients. Currently, there is no oral solution for Tacrolimus available on the market. For Prograf® the oral capsule options include 0.5, 1 and 5 mg dosages or intravenous administration. The U.S Food and Drug Administration recently approved an oral suspension form of Prograf® wherein the contents of unit dose packets containing solid granules comprising either 0.2 mg or 1 mg of Tacrolimus are suspended in water prior to administration. Because Tacrolimus has a narrow therapeutic index, it can be difficult to treat patients with the current oral dosage forms. Precise dosing is difficult to achieve with the current oral dosage forms and if more precise titration is required, the current dosage forms are intravenous. In addition, due to the complications of transplant operations, patients are oftentimes unable to swallow capsules. When this occurs, patients may be admitted to the hospital to receive Tacrolimus intravenously, which presents difficulties if there is no other reason for the patient to be admitted, or the patient may receive a compounded suspension from the pharmacy, which may offer inconsistent dosing if not shaken appropriately. The oral suspension also presents issues because of potential adverse mouth feel associated with the suspended particles and the possibility of inaccurate dosing if portions of the suspended Tacrolimus remain (adhered) to the dosing cup or dosing syringe in which the oral suspension is created and administered.

The formulations of the present disclosure provide advantages over the currently available solid oral and powder for oral suspension dosage forms. It would be desirable to provide formulations, dosage forms and methods that allow for the flexibility of dosing, dose titration and can ease the oral administration to patients, including pediatric and geriatric patients. It would be desirable to provide a formulation, method, kit and dosage form for an oral pharmaceutical formulation that can be used for the treatment of organ and stem cell transplant recipients and for the treatment of other T-cell mediated diseases, and can provide improved solubility, stability and consistent bioavailability. It is also desirable to provide an oral liquid formulations with acceptable taste and mouthfeel, which do not require reconstitution, mixing and/or dilution prior to administration.

### SUMMARY

The present disclosure relates to formulations, methods, kits, and dosage forms of an oral pharmaceutical formulation for treating organ or stem cell transplant recipients or T-cell mediated diseases. In a first exemplary embodiment, the present disclosure provides a pharmaceutical formulation comprising one or more immunosuppressive agents and one or more solubilizers. In an embodiment, the pharmaceutical formulation further comprises one or more dispersants and one or more sparging agents.

In a further exemplary embodiment, the present disclosure provides a kit comprising one or more dosage forms and instructions for administering the dosage forms to a subject in need, wherein the dosage forms comprise a formulation for treating an organ or stem cell transplant recipient or a T-cell mediated disease, comprising one or more immunosuppressive agents and one or more solubilizers. In an embodiment, the formulation further comprises one or more dispersants and one or more sparging agents.

In yet a further exemplary embodiment, the present disclosure provides methods for treating an organ or stem cell transplant recipient, for preventing or lowering the risk of organ rejection, or for treating a T-cell mediated disease. The methods can comprise administering to a subject in need a pharmaceutical formulation according to the present disclosure.

In yet another exemplary embodiment, the present disclosure provides methods of manufacturing or stabilizing an oral pharmaceutical formulation for the treatment of an organ or stem cell transplant recipient or a T-cell mediated disease. The methods can comprise mixing one or more solubilizers with one or more dispersants with continuous sparging, and adding one or more immunosuppressive agents with continuous sparging to obtain a clear solution.

In yet another exemplary embodiment, the present disclosure provides a dosage form comprising a pharmaceutical formulation for the treatment of an organ or stem cell transplant recipient or a T-cell mediated disease. The dosage forms can comprise a pharmaceutical formulation according to the present disclosure.

In yet another exemplary embodiment, the present disclosure provides an oral liquid dosage formulation for the treatment of an organ or stem cell transplant recipient or a T-cell mediated disease wherein the oral liquid dosage formulation comprises: (a) one or more immunosuppressive agents and (b) one or more solubilizers for the immunosuppressive agents. In certain embodiments the oral liquid dosage formulation further comprise one or more pharmaceutically acceptable excipients selected from the group consisting of flavoring agents, preservatives, buffering agents, pH adjusting agents, dispersants, carriers or combinations thereof. In certain embodiments the oral liquid dosage formulation is: (i) a solution; (ii) non-aqueous or substantially water free; (iii) free or substantially free of any low molecular weight mono alcohols; (iv) free or substantially free of any dispersants such as surfactants or wetting agents; (v) free or substantially free of any emulsifying agent; and (vi) any combination of (i), (ii), (iii), (iv) and (v).

In yet another exemplary embodiment, the present disclosure provides an oral liquid dosage formulation for the treatment of an organ or stem cell transplant recipient or a T-cell mediated disease that is “ready to use” by simply measuring the appropriate volume of the oral liquid into a dosing device such as a cup, spoon or syringe without further manipulation such as suspending, diluting or combining with additional components. In a further embodiment of the ready to use oral liquid dosage formulation, the oral liquid may not require shaking, mixing, and/or stirring prior to use.

In yet another exemplary embodiment, the present disclosure provides an oral liquid dosage formulation for the treatment of an organ or stem cell transplant recipient or a T-cell mediated disease that can be stored for at least three months or longer under refrigerated conditions (i.e., between 0° C. and 10° C.). Alternatively, the oral liquid dosage formulation may be stored at ambient conditions or room temperature, i.e. without the need for refrigeration for three months or longer.

### DETAILED DESCRIPTION

The following detailed description is exemplary and explanatory and is intended to provide further explanation of the present disclosure described herein. Other advantages, and novel features will be readily apparent to one of ordinary skill in the art from the following detailed description of the present disclosure.

The present disclosure provides one or more oral pharmaceutical formulations comprising at least one immunosuppressive agent or non-immunosuppressive FK binding protein ligand. The pharmaceutical formulation can further comprise additional components, for example one or more dispersants, solubilizers and/or sparging agents. In one embodiment, the present disclosure comprises novel formulations comprising an immunosuppressive compound as the active ingredient. The formulations described herein are useful in organ and stem cell transplant recipients and other T-cell mediated diseases in patients by administering one or more of the formulations to patients in need thereof. The formulations described herein are particularly desirable because of their unexpected superior solubility and stability profiles over a predetermined amount of time and because of their efficacy.

Any suitable immunosuppressive agent can be used in the present pharmaceutical formulations, including: a calcineurin inhibitor (e.g., cyclosporin (CsA) and analogs thereof, ISA(TX) 247, and Tacrolimus); azathioprine (AZ); mycophenolate mofetil (MMF); mizoribine (MZ); leflunomide (LEF); adrenocortical steroids (also known as adrenocortical hormones, corticosteroids, or corticoids) such as prednisolone and methylprednisolone; sirolimus (also known as rapamycin); everolimus; FK778; TAFA-93; deoxyspergualin (DSG); and 2-amino-2-[2-(4-octylphenyl)ethyl]-1,3-propanediol hydrochloride (FTY720).

Other suitable immunosuppressive agents include: cyclophosphamide; 15-deoxyspergualin (Gusperimus); interferons; sulfasalazine; mimoribine; misoprostol; anti-IL-2 receptor antibodies; thalidomide; anti-tumor necrosis factor antibodies; anti-CD2 antibodies; anti-CD147 antibodies; anti-CD4 antibodies; anti-CD8 antibodies and anti-thymocyte globulin antibodies; ORTHOCLONE® (also known as OKT3, from Ortho Biotech, Raritan, N.J.); SANDIMMUNE® ORAL (cyclosporine), available for example from Sandoz Pharmaceuticals, Hanover, N.J.; PROGRAF®, also known as Tacrolimus, available for example from Fujisawa Pharmaceuticals, Deerfield, Ill.); CELLCEPT®, also known as mycophenolate, available for example from Roche Pharmaceuticals, Nutley, N.J.; and RAPAMUNE®, also known as sirolimus, available for example from Pfizer, Inc., Collegeville, Pa.). In some embodiments, the immunosuppressive agent is rapamycin, Tacrolimus, mycophenolic acid, azathioprine or cyclophosphamide. Still other suitable immunosuppressive agents include an interleukin-2 alpha-chain blocker (e.g., basiliximab and daclizumab); an inhibitor of inosine monophosphate dehydrogenase (e.g., mycophenolate mofetil); or an inhibitor of dihydrofolic acid reductase (e.g., methotrexate). In one embodiment of the present disclosure, the immunosuppressive agent is Tacrolimus.

The pharmaceutical formulations of the present disclosure can comprise at least one FK binding protein ligand. Examples include FK-506 (Tacrolimus) and derivatives/analogs thereof, including 506BD and L0685,818; rapamycin and derivatives/analogs thereof including Way-124466, RAD001, CCI-779, and AP23573; ascomycin and derivatives/analogs thereof including pimecrolimus. See, e.g., Liu et al., 23(11) EXPERT OPIN. THER. PATENTS 1435-49 (2013), the entire disclosure of which is herein incorporated by reference. Furthermore, although the immunosuppressive agent Tacrolimus/FK-506 is an FK binding protein ligand, in certain embodiments, an FK binding protein ligand can comprise a non-immunosuppressive FK binding protein ligand. Examples of non-immunosuppressive ligands include meridamycin, antascomicins, and synthetic ligand of FKBP (SLF).

The words “comprise”, “comprises”, and “comprising” are to be interpreted inclusively rather than exclusively. The words “consist”, “consisting”, and its variants, are to be interpreted exclusively, rather than inclusively.

As used herein, the term “about” means a variability of 10% from the reference given, unless otherwise specified.

As used herein, the term “free” means the composition, formulation or material does not contain the component modified by the term “free”.

As used herein, the term “substantially free” means the composition, formulation or material contains small amounts of the component modified by the term “substantially free”. For example, a formulation that is substantially free of ethanol may contain less than 5.0%, 4.75%, 4.5%, 4.25%, 4.0%, 3.75%, 3.5%, 3.25%, 3.0%, 2.75%, 2.5%, 2.25%, 2.0%, 1.75%, 1.5%, 1.25%, 1.0%, 0.95%, 0.90%, 0.85%, 0.80%, 0.75%, 0.70%, 0.65%, 0.60%, 0.55%, 0.50%, 0.45%, 0.40%, 0.35%, 0.30%, 0.25%, 0.20%, 0.15%, or 0.10% weight percent ethanol.

As used herein the term “non-aqueous” should be accorded its normal meaning which may mean the composition, formulation or material does not contain any added or additional water other than the amount of water that commonly associated with the non-water components present in the composition, formulation or material. For example, a non-aqueous composition comprising solvents such as glycerin or propylene glycol will contain trace amounts of water because the United States Pharmacopeia allows glycerin to contain up to 5.0% water and propylene glycol to contain up to 0.2% water.

As used herein the term “low molecular weight” includes straight or branched C₁-C₁₂, preferably straight or branched C₁-C₈ and most preferably straight or branched C₁-C₆ chains.

A “patient” or “subject” is a mammal, e.g., a human or a veterinary patient or subject, e.g., mouse, rat, guinea pig, dog, cat, horse, cow, pig, or non-human primate, such as a monkey, chimpanzee, baboon or gorilla. In certain embodiments, the pharmaceutical formulations of the present disclosure can be administered to an adult patient or a pediatric patient. In one embodiment, the pediatric patient can be between the ages of six months to ten years.

The term “treating” or “treatment” is meant to encompass administering to a subject a compound of the present disclosure for the purposes of amelioration of one or more symptoms of a disease or disorder, including palliative care. As used herein, an “effective amount” or a “therapeutically effective amount” is used interchangeably and refers to an amount of a pharmaceutical formulation of the present disclosure which provides the desired treatment of a subject. As would be appreciated by one of ordinary skill in the art, the therapeutically effective amount of the present pharmaceutical formulations to treat a given disease, disorder or condition will vary from subject to subject, depending on factors such as age, general condition of the subject, the severity of the condition being treated, the particular compound and/or formulation administered, and the like. An appropriate therapeutically effective amount of the present pharmaceutical formulations suitable for any individual subject can be readily determined by one of ordinary skill in the art from the information provided herein.

The pharmaceutical formulations of the present disclosure are in biologically compatible form suitable for administration to subjects, for example to humans. The pharmaceutical formulations can further comprise a pharmaceutically acceptable excipient. The term “pharmaceutically acceptable” means suitable for use in humans or animals, for example as approved by a governmental regulatory agency (such as the US Food and Drug Administration) or listed in the U.S. Pharmacopeia (USP) or other generally recognized pharmacopeia, or which are generally recognized as safe (GRAS).

In an embodiment, pharmaceutical formulations of the present disclosure comprise one or more dispersants. The one or more dispersants can be any suitable dispersant, for example Polysorbate 80. Dispersants may include excipients such as surfactants and wetting agents.

In an embodiment, pharmaceutical formulations of the present disclosure comprise one or more solubilizers. A list of potential solubilizers are provided on pages 3258 and 3261 of USP 29 (2006) which are incorporated herein by reference. Some examples of solubilizers may include excipients such as emulsifying agents, surfactants, wetting agents, organic solvents, oils and combinations thereof. Examples of emulsifying agents, surfactants and wetting agents that maybe used as solubilizers are provided in greater detail below but some of the more preferred examples include but are not limited to polyoxyethylene sorbitan fatty acid esters, such as polysorbate 20, 40, 60 or 80, polyoxyethylene alkyl ethers, sorbitan esters, phosphatidylcholine, cyclodextrin and combinations thereof. Examples of organic solvents that may be used include but are not limited to low molecular weight mono alcohols such as ethanol; polyols, such as low molecular weight aliphatic triols, examples of which include glycerin and low molecular weight aliphatic diols, examples of which include propylene glycol; and polyethers, such as polyoxyalkylenes, examples of which include polyethylene glycols (“PEG”), preferably having a molecular weight less than 1000, less than 800 or less than 600. Examples of oils that may be used include, mineral oil, castor oil, vegetable and nut oils such as sunflower oil, corn oil, peanut oil, cottonseed oil, sesame oil, olive oil, canola oil, almond oil, safflower oil, soybean oil and combinations of the foregoing. Triacetin may also be used as a solubilizer in the dosage forms of the present invention. In certain embodiments, the solubilizer should be a liquid are room temperature. In certain embodiments, the solubilizer may be selected from the group consisting of polyethylene glycols, propylene glycol, glycerin, triacetin, ethanol, polysorbates, oils and combinations thereof.

In an embodiment, pharmaceutical formulations of the present disclosure comprise one or more sparging agents. The one or more sparging agents can be any suitable sparging agents, for example, any inert gas, including nitrogen, argon, carbon dioxide etc. In one embodiment, the one or more dispersants can be any suitable dispersing agents, e.g. (without limitation), Polysorbate 80, Polysorbate 20, Poloxamers etc. In one embodiment, one or more solubilizing agents can be any suitable solubilizer, e.g. (without limitation), cyclodextrin, glycerides, phospholipids etc.

As used herein, the term “excipient” refers to an inactive ingredient with which the immunosuppressive agent is administered. Pharmaceutically acceptable excipients are described in various reference materials such as the USP 29 (2008) and Rowe et al., eds., Handbook of Pharmaceutical Excipients, 7th Edition, London: Pharmaceutical Press, 2012. Some examples of suitable pharmaceutically acceptable excipients can be sterile liquids, such as water and oils, including those of petroleum, animal, vegetable or synthetic origin such as peanut oil, soybean oil, mineral oil, sesame oil and the like. Water can be a pharmaceutically acceptable excipient when the pharmaceutical formulation is administered orally or parenterally. Other suitable pharmaceutically acceptable excipients include starch, glucose, lactose, sucrose, gelatin, malt, rice, flour, chalk, silica gel, sodium stearate, glycerol monostearate, talc, sodium chloride, dried slim milk, glycerol, propylene, glycol, water, ethanol and the like. The pharmaceutical formulation can also contain minor amounts of wetting or emulsifying agents, or pH buffering agents.

The pharmaceutical formulations of the present disclosure can take any suitable form for oral administration to a subject, such as a human subject, for example solutions, suspensions, emulsions, tablets, pills, capsules, powders, sustained-release formulations and the like. Oral pharmaceutical formulations of the disclosure can include standard carriers as pharmaceutical excipients, such as pharmaceutical grades of mannitol, lactose, starch, magnesium stearate, sodium saccharine, cellulose, magnesium carbonate, etc. In a specific embodiment, a pharmaceutical formulation of the disclosure comprises an effective amount of an immunosuppressive agent together with a suitable amount of one or more pharmaceutically acceptable excipients so as to provide the form for proper administration to the patient, for example by oral administration via a solution, suspension, capsule, tablet, powder, or pill. For a discussion of the properties of solid and liquid pharmaceutically acceptable excipients which are suitable for use in the present pharmaceutical formulations, see, e.g., the excipients described in the Rowe et al., eds., Handbook of Pharmaceutical Excipients, 7th Edition, London: Pharmaceutical Press, 2012, which is incorporated herein by reference.

In an alternative embodiment, the pharmaceutical formulations of the present disclosure are oral liquid dosage formulations for the treatment of an organ or stem cell transplant recipient or a T-cell mediated disease wherein the oral liquid dosage formulation comprises:

(a) an immunosuppressive agent; and

(b) at least one pharmaceutically acceptable solubilizer as previously described.

In certain embodiments the solubilizer is a liquid at room temperature. In alternative embodiments the solubilizer is selected from the group consisting of polyethylene glycols, propylene glycol, glycerin, triacetin, ethanol, polysorbates, oils and combinations thereof.

The oral liquid dosage formulation may comprise from about 0.001 wt % to about 25 wt % of the immunosuppressive agent or agents, preferably about 0.01 wt % to about 20 wt % of the immunosuppressive agent or agents and most preferably about 0.05 wt % to about 15 wt % of the immunosuppressive agent or agents.

The oral liquid dosage formulation may comprise one or more pharmaceutically acceptable solubilizers. Preferably the oral liquid will comprise at least 0.5, 1, 5, 10, 15, 20, 25, 30, 35 40, 45, 50, 55, 60, 65, 70, 75, 80, 85, 90, or 95% w/v of one or more solubilizers as previously described. In certain embodiments, the total amount of one or more solubilizers employed in the oral liquid dosage formulations will comprise from about 0.5% w/v to about 99.999% w/v of the total weight of the liquid dosage formulation, about 1% w/v to about 99.99% w/v of the total weight of the liquid dosage formulation, about 5% w/v to about 99.999% w/v of the total weight of the liquid dosage formulation, preferably about 15% w/v to about 99.999% w/v of the total weight of the liquid dosage formulation and most preferably about 30% w/v to about 99.999% w/v of the total weight of the liquid dosage formulation.

In certain embodiments the oral liquid dosage formulation further comprise one or more pharmaceutically acceptable excipients selected from the group consisting of flavoring agents, preservatives, sweeteners, buffering agents, pH adjusting agents, dispersants, carriers or combinations thereof.

Examples of flavoring agents that may be employed in the solid dosage form of the present invention include artificial sweeteners such as aspartame, sucralose, saccharin, dipotassium glycyrrhizinate, stevia, thaumatin, and flavorants such as citric acid, peppermint oil, wintergreen oil, menthol, lemon, lime, orange, grape, cherry, and vanilla extract and combinations of the foregoing. Additional flavoring agents are described in U.S. Pat. No. 6,027,746 which is incorporated herein by reference. The total amount of flavoring agent or agents will range from about 0.01 wt % to about 20 wt % of the liquid dosage formulation, preferably about 0.05 wt % to about 15 wt % of the liquid dosage formulation and most preferably about 0.01 wt % to about 10 wt % of the liquid dosage formulation.

Preservatives that may be used in the liquid dosage formulations include, antioxidants, antimicrobial agents, chelating agents and combinations thereof.

Examples of antioxidants that may be employed include ascorbic acid, ascorbyl palmitate, butylated hydroxyanisole, butylated hydroxytoluene, hypophosphorous acid, monothioglycerol, potassium metabisulfate, propyl gallate, sodium bisulfite, sodium formaldehyde sulfoxylate, sodium metabisulfate, sodium sulfate, sodium thiosulfate, sodium dioxide, tocopherol, and mixtures thereof. The antioxidant or antioxidants may be present in the liquid dosage formulations of the present invention in an amount from about 0.001 wt % to about 10 wt % of the liquid dosage formulation, preferably about 0.005 wt % to about 5 wt % of the liquid dosage formulation and most preferably about 0.01 wt % to about 2.5 wt % of the liquid dosage formulation.

Examples of antimicrobial agents that may be employed include benzalkonium chloride, benzethonium chloride, benzoic acid, benzyl alcohol, butylparaben, cetrimonium bromide, cetylpyridinium chloride, chlorobutanol, chlorocresol, ethylparaben, methylparaben, phenol, phenoxyethanol, phenylethyl alcohol, potassium benzoate, potassium sorbate, propylparaben, sodium benzoate, sodium dehydroacetate, sodium propionate, sorbic acid, thimersol, thymol and combinations thereof. The antimicrobial agent or agents may be present in the liquid dosage formulations of the present invention in an amount from about 0.001 wt % to about 5 wt % of the liquid dosage formulation, preferably about 0.005 wt % to about 2.5 wt % of the liquid dosage formulation and most preferably about 0.01 wt % to about 1.0 wt % of the liquid dosage formulation.

Examples of chelating agents that may be employed include citric acid and salts thereof, polyphosphates (e.g., sodium tripolyphosphate, hexametaphosphoric acid, sodium acid pyrophosphate, sodium pyrophosphate, tetra sodium pyrophosphate, sodium hexametaphosphate, sodium metaphosphate); aminocarboxylic acids (e.g., ethylenediaminetetraacetic acid (EDTA), 1,2-bis(2-amino-phenoxy)ethane-N,N,N′N′-tetraacetic acid (EGTA), ethylenebis(oxyethylenenitrilo)tetraacetic acid (BAPTA)) and salts thereof. The chelating agent or agents may be present in the liquid dosage formulations of the present invention in an amount from about 0.001 wt % to about 5 wt % of the liquid dosage formulation, preferably about 0.005 wt % to about 2.5 wt % of the liquid dosage formulation and most preferably about 0.01 wt % to about 1.0 wt % of the liquid dosage formulation.

Examples of buffering agents that may be employed include acetic acid, adipic acid, ammonium carbonate, ammonium phosphate, boric acid, citric acid, lactic acid, phosphoric acid, potassium citrate, potassium phosphate, sodium acetate, sodium citrate, sodium carbonate, potassium carbonate, sodium bicarbonate, potassium bicarbonate, sodium lactate, sodium phosphate, succinic acid, and combinations thereof. Typically the buffer will comprise a combination of the foregoing as to create a buffer system such as citric acid and sodium citrate or acetic acid and sodium acetate. The buffering agent or agents may be present in the liquid dosage formulations of the present invention in an amount from about 0.01 wt % to about 10 wt % of the liquid dosage formulation, preferably about 0.05 wt % to about 5 wt % of the liquid dosage formulation and most preferably about 0.1 wt % to about 2.5 wt % of the liquid dosage formulation.

Examples of pH adjusting agents that may be employed include, but are not limited to, any of the pharmaceutically acceptable acids or bases used to adjust the pH of pharmaceutical compositions. Examples of compounds typically used to adjust the pH of pharmaceutical compositions include hydrochloric acid, citric acid, lactic acid, tartaric acid, glacial acetic acid, sodium hydroxide, potassium hydroxide, arginine, lysine meglamine, triethanol amine, or combinations thereof. The amount of pH adjusting agents may be present in the liquid dosage formulations to obtain a desired pH, typically from 3-8.

Some additional examples of dispersants and solubilizing agents that may be employed in the oral liquid formulations have been previously identified. In certain embodiment the dispersants and solubilizers include emulsifying agents and surfactants. The surfactants further include non-ionic surfactants, ionic surfactants or a combination thereof. Examples of non-ionic surfactants include polyethoxylated castor oil, a polyoxyethylene alkyl ester, a polyglycolyzed glyceride, a sorbitan fatty acid ester, a glycerin fatty acid ester, a fatty acid polyglyceride, a fatty acid alcohol polyglycol ether, acetylene glycol, acetylene alcohol, an oxyalkylene block polymer, a polyoxyethylene alkyl ether, a polyoxyethylene alkylaryl ether, a polyoxyethylene styrylaryl ether, a polyoxyethylene glycol alkyl ether, a polyoxyethylene fatty acid ester, a polyoxyethylene sorbitan fatty acid ester, a polyoxyethylene glycerin fatty acid ester, a polyoxyethylene hydrogenated castor oil, a polyoxypropylene fatty acid ester, or a mixture of the foregoing. A further listing of possible non-ionic surfactants can be found on pages 1243-1249 of Martindale, The Extra Pharmacopoeia 29^{th} ed. which is incorporated herein by reference. Certain non-ionic surfactants include polyoxyethylene derivatives of polyol esters, such as Polysorbate 20 (TWEEN 20®), Polysorbate 40 (TWEEN 40®) Polysorbate 60 (TWEEN 60®), and Polysorbate 80 (TWEEN 80®), d-alpha tocopheryl polyethylene glycol 1000 succinate (TPGS), nonoxinols, poloxamers, sorbitan monolaurate, sorbitan monooleate, sorbitan monopalmitate, sorbitan monostearate, sorbitan sesquioleate, sorbitan trioleate, tyloxapol, and mixtures of the foregoing.

Ionic surfactants include, but are not limited to, carboxylates such as phospholipids, soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, phosphates, quaternary ammonium salts, and ethoxylated amines.

In certain embodiments, the dosage forms may further include a carrier. In the oral liquid dosage formulations, the carriers may include liquid excipients and diluents such as pharmaceutically acceptable solvents and co-solvents, preferably solvents and co-solvents that may be orally ingested. Examples of the liquid carriers have been described above and may in certain embodiments include or exclude water. The carriers may also include amounts of the foregoing solubilizers and dispersants that exceed the amounts required to dissolve or disperse the immunosuppressive agents in the liquid formulation.

In certain embodiments the oral liquid dosage formulations is solution, suspension or dispersion and most preferably a solution.

In certain embodiments the oral liquid dosage formulations is a non-aqueous solution, or a solution substantially free of water.

In certain embodiments the oral liquid dosage formulations is a solution that is free or substantially free any low molecular weight mono alcohols such as ethanol.

In certain embodiments the oral liquid dosage formulations is a solution free or substantially free of any surfactant.

In certain embodiments the oral liquid dosage formulations is a solution free or substantially free of an emulsifying agent.

In certain embodiments the oral liquid dosage formulations is a solution comprising (a) an immunosuppressive agent; (b) one or more pharmaceutically acceptable solubilizers selected from the group consisting of polyethylene glycols, propylene glycol, glycerin, triacetin, oils and combinations thereof; (c) optionally one or more pharmaceutically acceptable excipients selected for the group consisting of flavoring agents, preservatives, buffering agents, pH adjusting agents, carriers or combinations thereof and wherein the oral liquid dosage formulation is non-aqueous, substantially free of low molecular weight mono alcohols such as ethanol, substantially free of any surfactant and substantially free of any emulsifier.

In some embodiments, the pharmaceutical formulations of the disclosure comprise an immunosuppressive agent that can be used alone, e.g., an oral formulation comprising an immunosuppressive agent without any other active ingredient, or in concert with at least one other active ingredient at appropriate dosages of the at least one other active ingredient as are known in the art to achieve a desired treatment, for example as defined by routine testing in order to obtain optimal efficacy while minimizing any potential toxicity.

Suitable therapeutically effective amounts and dosage regimens utilizing a pharmaceutical formulation of the disclosure can be selected by the ordinarily skilled clinician in accordance with a variety of factors, including species, age, weight, sex, and overall medical condition of the patient; the condition to be treated and its severity or penetration; the route of administration; the renal and hepatic function of the patient; and the particular pharmaceutical formulation employed.

In some embodiments, the dosing for the immunosuppressive agent can be weight based and titrated based on a patient's blood levels. In certain embodiments, the immunosuppressive agent comprising pharmaceutical formulations of the disclosure can be administered in low dose amount. The phrase “low dose” or “low dose amount” of an immunosuppressive agent in the context of the present disclosure refers to the use of a particular amount of an immunosuppressive drug that is lower than typically used for immunosuppression, for example lower than typically used, or commercially available, for immunosuppression in a human. In one embodiment, the low dose amount refers to the use of a particular amount that is lower than typically used, or commercially available, for immunosuppression of a human organ transplant recipient that is calculated to prevent rejection.

In certain embodiments a low dose of an immunosuppressive agent, for example Tacrolimus, is less than about ⅕, ⅙, 1/7, ⅛, 1/9, 1/10, 1/11, 1/12, 1/13, 1/14, or less then about 1/15 of a normal dose used for immunosuppression in humans. In certain embodiments, the low dose of an immunosuppressive agent, for example Tacrolimus, is about or less than about 1/10 of the amount used for immunosuppression in humans.

In other embodiments, the low dose of the immunosuppressive agent, for example Tacrolimus, is about or less than ½, ⅓, ¼, ⅕, ⅙, 1/7, ⅛, or about or less than about 1/9 of the amount used for immunosuppression in humans. In further embodiments, the low dose of the immunosuppressive agent, for example Tacrolimus, is about or less than about 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, 0.009, 0.08, or 0.07 times than the typical amount used for a particular situation in humans to generate immunosuppression.

In other embodiments, the daily dose of the immunosuppressive agent, for example Tacrolimus, will range from about 0.01 mg/kg/day to about 1 mg/kg/day with the total daily dose being administered in divided doses such as two, three or four times a day. The following table provides some examples of the desired dosing:

| | |
|---|---|
| Adult Kidney | 0.1-0.2 mg/kg/day administered in two |
| Transplant Patients | doses every 12 hours |
| Adult Liver | 0.1-0.15 mg/kg/day administered in two |
| Transplant Patients | doses every 12 hours |
| Adult Heart | 0.075 mg/kg/day administered in two |
| Transplant Patients | doses every 12 hours |
| Pediatric Kidney | 0.3 mg/kg/day administered in two doses |
| Transplant Patients | every 12 hours |
| Pediatric Liver | 0.15-0.2 mg/kg/day administered in two |
| Transplant Patients | doses every 12 hours |
| Pediatric Heart | 0.3 mg/kg/day administered in two doses |
| Transplant Patients | every 12 hours |

The above dose may be adjusted based on other conditions such as renal or hepatic impairment levels.

In certain embodiments, pharmaceutical formulations of the present disclosure comprise a low dose of an immunosuppressive agent (e.g., Tacrolimus) in humans of about 0.01% w/v to about 1.0% w/v, about 0.01% w/v to 0.90% w/v, about 0.01% w/v to about 0.80% w/v, about 0.01% w/v to about 0.70% w/v, about 0.01% w/v to about 0.60% w/v, about 0.01% w/v to about 0.50% w/v, about 0.01% w/v to about 0.40% w/v, about 0.01% w/v to about 0.30% w/v, about 0.01% w/v to about 0.20% w/v, about 0.01% w/v to about 0.10% w/v, and about 0.01% w/v to about 0.05% w/v. In one embodiment, the low dose of Tacrolimus in humans is about 0.05% w/v to 0.1% w/v. However, the therapeutically effective amount of the immunosuppressive agent can be determined by the attending physician and may depend on the target therapeutic blood level to achieve immunosuppression without inducing toxicity, as well as the condition treated, the compound administered, the route of delivery, the age, weight, severity of the patient's symptoms and response pattern of the patient. In one embodiment, the immunosuppressive agent may be administered in an amount that achieves immunosuppression without inducing negative effects of a low therapeutic blood level (e.g., rejection of an organ) or a high therapeutic blood level (e.g., toxicity).

In further embodiments, pharmaceutical formulations of the present disclosure comprise an immunosuppressive agent (e.g., Tacrolimus) provided in a concentration of about 0.01 mg/ml to about 10 mg/ml. The concentration can be less than about 10 mg/ml, 9 mg/ml, 8 mg/ml, 7 mg/ml, 6 mg/ml, 5 mg/ml, 4 mg/ml, 3 mg/ml, 2 mg/ml, 1.0 mg/ml, 0.9 mg/ml, 0.8 mg/ml, 0.7 mg/ml, 0.6 mg/ml, 0.5 mg/ml, 0.4 mg/ml, 0.3 mg/ml, 0.2 mg/ml, 0.1 mg/ml, 0.09 mg/ml, 0.08 mg/ml, 0.07 mg/ml, 0.06 mg/ml, 0.05 mg/ml, 0.04 mg/ml, 0.03 mg/ml, 0.02 mg/ml or 0.01 mg/ml. In one embodiment, the immunosuppressive agent (e.g., Tacrolimus) is provided in a concentration of about 0.1 to about 1.5 mg/ml, or about 0.5 to about 1.0 mg/ml. In an embodiment, the immunosuppressive agent (e.g., Tacrolimus) is provided in a concentration of about 0.5 mg/ml. In another embodiment, the immunosuppressive agent (e.g., Tacrolimus) is provided in a concentration of about 1.0 mg/ml.

In certain embodiments the one or more dispersants (e.g., Polysorbate 80) can be provided in any suitable amount to achieve the purposes disclosed herein. In one embodiment, the one or more dispersants (e.g., Polysorbate 80) can be provided in an amount of about 0.5% w/v to about 1.5% w/v, about 0.6% w/v to about 1.4% w/v, about 0.7% w/v to about 1.3% w/v, about 0.8% w/v to about 1.2% w/v, and about 0.9% w/v to about 1.1% w/v. In one embodiment, the one or more dispersants (e.g., Polysorbate 80) is provided in the amount of about 0.8, 1.0, or 1.2% w/v.

In certain embodiments the one or more solubilizers (e.g., phosphatidylcholine, or cyclodextrin) can be provided in any suitable amount to achieve the purposes disclosed herein. In one embodiment, the one or more solubilizers (e.g., phosphatidylcholine, or cyclodextrin) can be provided in an amount of about 90% w/v to about 99.9% w/v, about 95% w/v to about 99.9% w/v, about 97% w/v to about 99.9% w/v, about 99.0% w/v to about 99.9% w/v, about 99.1% w/v to about 99.8% w/v, about 99.2% w/v to about 99.7% w/v about 99.3% w/v to about 99.6% w/v about 99.4% w/v to about 99.5% w/v, and about 99.40% w/v to about 99.45% w/v. In one embodiment, the one or more solubilizers (e.g., phosphatidylcholine, or cyclodextrin) is provided in the amount of about 99.45% w/v. In another embodiment, the one or more solubilizers (e.g., phosphatidylcholine, or cyclodextrin) is provided in the amount of about 99.20, 99.25, 99.40, 99.45, 99.60, or 99.65, % w/v.

The one or more sparging agents (e.g., inert gas, nitrogen) can be provided in any suitable amount to achieve the purposes described herein, for example in an amount to achieved the desired total volume.

As described above, FK506 (Tacrolimus) was discovered in 1987 from a type of soil bacterium, Streptomyces tsukubaensis. FK506 reduces peptidyl-prolyl isomerase activity by binding to the immunophilin FKBP12 (FK506 binding protein) creating a new complex. This FKBP12-FK506 complex interacts with and inhibits calcineurin, thus inhibiting both T-lymphocyte signal transduction and IL-2 transcription. FK506 was first approved by the FDA in 1994 for use in liver transplantation, and its uses have now been extended to include kidney, heart, small bowel, pancreas, lung, trachea, skin, cornea, bone marrow and limb transplants.

The therapeutically effective amounts may be provided on regular schedule, i.e., daily, weekly, monthly, or yearly basis or on an irregular schedule with varying administration days, weeks, months, etc. Alternatively, the therapeutically effective amount to be administered may vary. In one embodiment, the therapeutically effective amount for the first dose is higher than the therapeutically effective amount for one or more of the subsequent doses. In another embodiment, the therapeutically effective amount for the first dose is lower than the therapeutically effective amount for one or more of the subsequent doses. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. Alternatively, equivalent doses may be administered over uneven intervals in accordance with the recommended treatment of a health-care practitioner. The number and frequency of dosages corresponding to a completed course of therapy will be determined according to the judgment of a health-care practitioner. The therapeutically effective amounts described herein refer to total amounts administered for a given time period; that is, if more than one immunosuppressive agent is administered, the therapeutically effective amounts correspond to the total amount administered.

In certain embodiments, the pharmaceutical formulations of the disclosure may be administered at least once a week over the course of several weeks. In one embodiment, the pharmaceutical formulations are administered at least once a week over several weeks to several months. In another embodiment, the pharmaceutical formulations are administered once a week over four to eight weeks. In yet another embodiment, the pharmaceutical formulations are administered once a week over four weeks.

In other embodiments, the present pharmaceutical formulations can be administered at least once a day for about 2 days, at least once a day for about 3 days, at least once a day for about 4 days, at least once a day for about 5 days, at least once a day for about 6 days, at least once a day for about 7 days, at least once a day for about 8 days, at least once a day for about 9 days, at least once a day for about 10 days, at least once a day for about 11 days, at least once a day for about 12 days, at least once a day for about 13 days, at least once a day for about 14 days, at least once a day for about 15 days, at least once a day for about 16 days, at least once a day for about 17 days, at least once a day for about 18 days, at least once a day for about 19 days, at least once a day for about 20 days, at least once a day for about 21 days, at least once a day for about 22 days, at least once a day for about 23 days, at least once a day for about 24 days, at least once a day for about 25 days, at least once a day for about 26 days, at least once a day for about 27 days, at least once a day for about 28 days, at least once a day for about 29 days, at least once a day for about 30 days, or at least once a day for about 31 days.

In other embodiments, the present pharmaceutical formulations can be administered at least twice a day for about 2 days, at least twice a day for about 3 days, at least twice a day for about 4 days, at least twice a day for about 5 days, at least twice a day for about 6 days, at least twice a day for about 7 days, at least twice a day for about 8 days, at least twice a day for about 9 days, at least twice a day for about 10 days, at least twice a day for about 11 days, at least twice a day for about 12 days, at least twice a day for about 13 days, at least twice a day for about 14 days, at least twice a day for about 15 days, at least twice a day for about 16 days, at least twice a day for about 17 days, at least twice a day for about 18 days, at least twice a day for about 19 days, at least twice a day for about 20 days, at least twice a day for about 21 days, at least twice a day for about 22 days, at least twice a day for about 23 days, at least twice a day for about 24 days, at least twice a day for about 25 days, at least twice a day for about 26 days, at least twice a day for about 27 days, at least twice a day for about 28 days, at least twice a day for about 29 days, at least twice a day for about 30 days, at least twice a day for about 31 days, at least twice a day for one month, at least twice a day for two months, at least twice a day for three months, at least twice a day for four months, at least twice a day for five months, at least twice a day for six months or more, or at least twice a day for one year or more.

In other embodiments, the pharmaceutical formulations of the disclosure can be administered every other day for about 2 days, every other day for about 3 days, every other day for about 4 days, every other day for about 5 days, every other day for about 6 days, every other day for about 7 days, every other day for about 8 days, every other day for about 9 days, every other day for about 10 days, every other day for about 11 days, every other day for about 12 days, every other day for about 13 days, every other day for about 14 days, every other day for about 15 days, every other day for about 16 days, every other day for about 17 days, every other day for about 18 days, every other day for about 19 days, every other day for about 20 days, every other day for about 21 days, every other day for about 22 days, every other day for about 23 days, every other day for about 24 days, every other day for about 25 days, every other day for about 26 days, every other day for about 27 days, every other day for about 28 days, every other day for about 29 days, every other day for about 30 days, or every other day for about 31 days or more.

In other embodiments, the pharmaceutical formulations of the disclosure can be administered about once every day, about once every 2 days (also sometimes stated herein as once every other day), about once every 3 days, about once every 4 days, about once every 5 days, about once every 6 days, about once every 7 days, about once every 8 days, about once every 9 days, about once every 10 days, about once every 11 days, about once every 12 days, about once every 13 days, about once every 14 days, about once every 15 days, about once every 16 days, about once every 17 days, about once every 18 days, about once every 19 days, about once every 20 days, about once every 21 days, about once every 22 days, about once every 23 days, about once every 24 days, about once every 25 days, about once every 26 days, about once every 27 days, about once every 28 days, about once every 29 days, about once every 30 days, or about once every 31 days. In certain embodiments, the present pharmaceutical formulations can be administered every other day.

In other embodiments, the pharmaceutical formulations of the disclosure can be administered about once every week, about once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, about once every 12 weeks, about once every 13 weeks, about once every 14 weeks, about once every 15 weeks, about once every 16 weeks, about once every 17 weeks, about once every 18 weeks, about once every 19 weeks, or about once every 20 weeks.

In other embodiments, the pharmaceutical formulations of the disclosure can be administered about once every month, about once every 2 months, about once every 3 months, about once every 4 months, about once every 5 months, about once every 6 months, about once every 7 months, about once every 8 months, about once every 9 months, about once every 10 months, about once every 11 months, or about once every 12 months.

In other embodiments, the pharmaceutical formulations of the disclosure can be administered at least once a week for about 2 weeks, at least once a week for about 3 weeks, at least once a week for about 4 weeks, at least once a week for about 5 weeks, at least once a week for about 6 weeks, at least once a week for about 7 weeks, at least once a week for about 8 weeks, at least once a week for about 9 weeks, at least once a week for about 10 weeks, at least once a week for about 11 weeks, at least once a week for about 12 weeks, at least once a week for about 13 weeks, at least once a week for about 14 weeks, at least once a week for about 15 weeks, at least once a week for about 16 weeks, at least once a week for about 17 weeks, at least once a week for about 18 weeks, at least once a week for about 19 weeks, or at least once a week for about 20 weeks.

In other embodiments, the pharmaceutical formulations of the disclosure can be administered at least once a week for about 1 month, at least once a week for about 2 months, at least once a week for about 3 months, at least once a week for about 4 months, at least once a week for about 5 months, at least once a week for about 6 months, at least once a week for about 7 months, at least once a week for about 8 months, at least once a week for about 9 months, at least once a week for about 10 months, at least once a week for about 11 months, or at least once a week for about 12 months.

In certain embodiments, the oral pharmaceutical formulations of the disclosure can be administered in a dosing regimen or treatment method comprising administering the oral pharmaceutical formulations to a subject who has received an organ or stem cell transplant, including, without limitation, liver, kidney, small bowel, skin, heart, lung, trachea, cornea, bone marrow, limb, intestine, and pancreas. The treatment regimen can also be applied to composite tissue transplantation. The composite tissue can be hand, face, or any other anatomical part. In particular embodiments, the treatment regimen can be utilized for toxic liver injury such as acetaminophen or fulminant hepatitis. The formulations of the present disclosure can be useful in the treatment of patients with ischemic injury and/or shock. Although much of the present disclosure is made in the context of organ or stem cell transplantation, it should be recognized that the treatment regimens are broadly applicable, as noted above, and should not be construed as limited to organ or stem cell transplantation. Any suitable administration route can be used, for example via oral solution, suspension, tablets, capsules, powders or pills.

The treatment regimen of the present disclosure can recruit regulatory T-cells to the organ transplant site. Because regulatory T-cells are involved in controlling autoimmune diseases including, but not limited to, type 1 diabetes, experimental autoimmune encephalomyelitis, and inflammatory bowel disease, the mobilization of stem cells may have broader clinical applications rather than transplantation. In particular embodiments, therefore, the pharmaceutical formulations of the present disclosure can be used to treat various T-cell mediated autoimmune diseases. In one embodiment, the pharmaceutical formulations of the present disclosure can be used to treat atopic dermatitis, or other dermatologic conditions. Tacrolimus is a calcineurin-inhibitor immunosuppressant indicated for prophylaxis of organ rejection in patients receiving transplants, for example allogeneic liver, kidney or heart transplants. Tacrolimus can also be used concomitantly with adrenal corticosteroids or in kidney and heart transplants, used in conjunction with azathioprine or mycophenolate mofetil (MMF).

The pharmaceutical formulations of the present disclosure may be formulated neat or with one or more pharmaceutical excipients for administration. The amount of the pharmaceutical excipients(s) is determined by a number of factors and considerations including the solubility and chemical nature of the active ingredient (e.g., immunosuppressive agent), chosen route of administration and standard pharmacological practice. The pharmaceutical excipients(s) may be solid or liquid and may incorporate both solid and liquid excipients. A variety of suitable liquid excipients are known and have been described above and may be readily selected by one of skill in the art. Examples of excipients include, e.g., DMSO, saline, buffered saline, hydroxypropylcyclodextrin, and mixtures thereof. Similarly, a variety of solid excipients are known to those of skill in the art.

Although the immunosuppressive agent disclosed herein may be administered alone, it may also be administered in the presence of one or more pharmaceutical excipients that are physiologically compatible. The excipients may be in dry or liquid form and must be pharmaceutically acceptable. Liquid pharmaceutical formulations are typically sterile solutions or suspensions. When liquid excipients are utilized for parenteral administration, they are desirably sterile liquids. Liquid excipients are typically utilized in preparing solutions, suspensions, emulsions, syrups and elixirs. In one embodiment, the immunosuppressive agent is dissolved in a liquid excipient such as a solubilizer and may be further mixed with other liquid excipients such as additional solublizers, physiologically acceptable carries, solvents and combinations thereof. In another embodiment, the immunosuppressive agent is suspended in a liquid excipient such as water. One of skill in the art of formulations would be able to select a suitable liquid excipient, depending on the route of administration. The immunosuppressive agent may alternatively be formulated in a solid excipient. In one embodiment, the formulation may be compacted into a unit dose form, i.e., tablet or caplet. In another embodiment, the formulation may be added to unit dose form, i.e., a capsule. In a further embodiment, the formulation may be formulated for administration as a powder. The excipients may perform a variety of functions, i.e., may perform the functions of two or more of the excipients described below. For example, solid excipient may also act as a flavoring agent, lubricant, solubilizer, suspending agent, filler, glidant, compression aid, binder, disintegrant, or encapsulating material.

The formulation may also be sub-divided to contain appropriate quantities of the immunosuppressive agent. For example, the unit dosage can be packaged formulations, e.g., packeted powders, vials, ampoules, prefilled syringes, bottles or sachets containing liquids. In certain embodiments, the formulation is an oral liquid dosage form, preferably a ready to use dosage form that is packaged in any suitable container such as an amber glass bottle with a child resistant closure. The amber glass bottler may further comprise a press in bottle adapter, with or without a dip tube that will allow a user to withdraw the appropriate amount of the oral liquid dosage form into a dosing syringe. The packaged formulations may further include printed instructions describing the use and administration of the dosage form as well as dosing materials such as one or more dosing cups or one or more oral dosing syringes, with or without graduated volume markings. In certain embodiments the dosing cup or oral dosing syringe would comprise indicia indicated the volume of the dosage form in the dosing cup or oral dosing syringe, preferably in increments of 0.1 mL, 0.25 mL, 0.5 mL or combinations thereof.

In another embodiment, the formulations may be utilized as aerosols, i.e., oral or intranasal. For this route of administration, the formulations are formulated for use in a pressurized aerosol container together with a gaseous or liquefied propellant, e.g., dichlorodifluoromethane, carbon dioxide, nitrogen, propane, and the like. Also provided is the delivery of a metered dose in one or more actuations.

In another embodiment, the formulations may be administered by a sustained delivery device. “Sustained delivery” as used herein refers to delivery of the immunosuppressive agent which is delayed or otherwise controlled. Those of skill in the art know suitable sustained delivery devices. For use in such sustained delivery devices, the immunosuppressive agent is formulated as described herein.

In treatment methods of the disclosure, the injury which triggers administration of the first dose of the present pharmaceutical formulations can be any tissue injury that signals or indicates that a particular treatment is necessary. For example, the tissue injury can be an organ transplant (including liver, kidney, small bowel, skin, heart, lung, trachea, cornea, bone marrow, limb, intestine, and pancreas) or the diagnosis of a T-cell mediated disease or other autoimmune or inflammatory disease or the occurrence of an episode of a T-cell mediated disease or other autoimmune or inflammatory disease. For example, administration of the first dose can be immediately upon occurrence of the tissue injury, or as soon thereafter as is practical or medically feasible, for example on the same day that the tissue injury occurred or was observed, such as within about one minute, five minutes, thirty minutes, sixty minutes, ninety minutes, 2 hours, 3 hours, 4 hours, 5 hours, 10 hours, 12 hours or 18 hours after occurrence of the tissue injury. In some embodiments, administration of the first dose can be delayed from the occurrence of the tissue injury, for example by about one day, 2 days, 3 days, 4 days, 5 days, 6 days or 7 days.

The pharmaceutical formulations disclosed herein may comprise oral formulations having improved solubility and/or stability profiles. The formulations may be suitable for oral delivery, for example via a solution or suspension. The formulations orally administrated via a solution or suspension can be bioequivalent to formulations administered via oral capsules, or any other administration route. The formulations disclosed herein are stable under standard storage conditions or accelerated conditions. The total amount of impurities in the formulations may be not more than about 0.1 to 3%, and standard storage conditions may comprise a temperature of about 20 to 25° C. (i.e., room temperature) and no more than about 40% Relative Humidity (RH). In one embodiment, the formulations disclosed herein are stable at room temperature for 18 to 24 months or longer. The stability of a formulation according to the present disclosure can be determined, for example, by measuring the physical state of the formulation, including the viscosity and presence of any discoloration and chemical stability by measuring assay of API & related compound.

In some embodiments, the formulations of the disclosure are stable when subject to predetermined conditions for predetermined times. For example, pharmaceutical formulations of the disclosure can be stored at various predetermined temperatures and relative humidities for defined or predetermined time periods, for example in an open or closed container. In some embodiments, formulations of the disclosure are stable upon storage at about 0, 2, 5, 8, 10, 15, 20, 25, 30, 37, 40 or 45 degrees Celsius and about 0%, 5%, 10%, 15%, 20%, 25%, 30%, 35%, 40%, 45%, 50%, 55%, 60%, 65%, 70%, 75%, 80%, 85%, 90%, 95% or 100% relative humidity for a period of at least about 0.5, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 20, 25, 30, 35, 40, 45, 48, 50, 51, 52, 53, 55 or 60 hours 1 week, 2 weeks, 3 weeks or 4 week; 1 month, 2 months, 3 months, 4 months, 5 months or 6 months.

In some embodiments, formulations of the disclosure are stable upon storage in an open or closed container at: about 30 degrees Celsius and about 90 percent relative humidity for a period of at least about 20 hours; about 40 degrees Celsius and about 60 percent relative humidity for a period of at least about one week, two weeks or three weeks; about 40 degrees Celsius and about 75 percent relative humidity for a period of at least about one week, two weeks or three weeks; about 25 degrees Celsius and about 60 percent relative humidity for a period of at least about one month; about 40 degrees Celsius and about 75 percent relative humidity for a period of at least one month; about 25 degrees Celsius and about 60 percent relative humidity for a period of at least about 3 months; or 5 degrees Celsius at any relative humidity for a period of at least about three months. In some embodiments, “storage in an open container” means that the container was opened twice a day for a given period of time, for example up to four weeks, but was otherwise left closed.

In some embodiments, the formulations of the present disclosure are stable when subject to predetermined conditions for predetermined times. For example, pharmaceutical formulations of the present disclosure can be stored at various predetermined temperatures and relative humidities for defined or predetermined time periods, for example in an open or closed container. In some embodiments (see Table 1), formulations of the disclosure are stable upon storage at about 5° C.±3° C. for up to 12 months and no significant degradation was observed. The stability results are provided in Table 4. A composition (see Table 2) was also stored at Controlled Room temperature (CRT) (25° C./60% RH) for up to 3 months, and it exhibited good stability whereby the assay value was greater than 99%. However, the extent of impurities was increased as compared to the samples stored at 5° C.±3° C. (see Table 5).

In some embodiments, and particularly the oral liquid dosage formulations described in paragraphs [0033]-[0053] above, can be stored for at least three months, four months, five months, six months, or longer under refrigerated conditions, preferably at a temperature between about 0° C. and about 10° C. and more preferably between about 2° C. and about 8° C. when packaged in a single or multiple dose amber glass bottle with a conventional child resistant screw cap and with or without a press-in bottle adapter/orifice reducer with a dip tube. In certain embodiments the oral liquid dosage formulations will exhibit the following impurity profiles as determined by High Performance Liquid Chromatography analysis when stored under refrigerated conditions in an amber glass bottle with a conventional child resistant screw cap:

| | | Preferred | Most Preferred |
|---|---|---|---|
| | Amount | Amount | Amount |
| Individual Known | Less than | Less than | Less than |
| Impurity | 0.5% | 0.25% | 0.15% |
| Total Known | Less than | Less than | Less than |
| Impurities | 1% | 0.75% | 0.5% |
| Individual Unknown | Less than | Less than | Less than |
| Impurity | 0.5% | 0.4% | 0.3% |
| Total Unknown | Less than | Less than | Less than |
| Impurities | 1% | 0.75% | 0.5% |

In certain oral liquid dosage formulations comprising Tacrolimus, the stability profiles should be as follows:

| | | Preferred | Most Preferred |
|---|---|---|---|
| | Amount | Amount | Amount |
| Tacrolimus 8-Epimer | Less than | Less than | Less than |
| | 0.5% | 0.25% | 0.15% |
| Tacrolimus Diene | Less than | Less than | Less than |
| | 0.5% | 0.25% | 0.15% |
| Total Known | Less than | Less than | Less than |
| Impurities | 1% | 0.75% | 0.5% |
| Individual Unknown | Less than | Less than | Less than |
| Impurity | 0.5% | 0.4% | 0.3% |
| Total Unknown | Less than | Less than | Less than |
| Impurities | 1% | 0.75% | 0.5% |

In some embodiments, and particularly the oral liquid dosage formulations described in paragraphs [0033]-[0053] above, can be stored for at least three months, four months, five months, six months, or longer at ambient conditions or room temperature, i.e. without the need for refrigeration or under CRT conditions, when packaged in a single or multiple dose amber glass bottle with a conventional child resistant screw cap as described above. In certain embodiments the oral liquid dosage formulations will exhibit the following impurity profiles as determined by High Performance Liquid Chromatography analysis when stored at ambient conditions in an amber glass bottle with a conventional child resistant screw cap:

| | | Preferred | Most Preferred |
|---|---|---|---|
| | Amount | Amount | Amount |
| Individual Known | Less than | Less than | Less than |
| Impurity | 0.5% | 0.25% | 0.15% |
| Total Known | Less than | Less than | Less than |
| Impurities | 1% | 0.75% | 0.5% |
| Individual Unknown | Less than | Less than | Less than |
| Impurity | 0.5% | 0.4% | 0.3% |
| Total Unknown | Less than | Less than | Less than |
| Impurities | 1% | 0.75% | 0.5% |

In certain oral liquid dosage formulations comprising Tacrolimus, the stability profile should be as follows:

| | | Preferred | Most Preferred |
|---|---|---|---|
| | Amount | Amount | Amount |
| Tacrolimus 8-Epimer | Less than | Less than | Less than |
| | 0.5% | 0.25% | 0.15% |
| Tacrolimus Diene | Less than | Less than | Less than |
| | 0.5% | 0.25% | 0.15% |
| Total Known | Less than | Less than | Less than |
| Impurities | 1% | 0.75% | 0.5% |
| Individual Unknown | Less than | Less than | Less than |
| Impurity | 0.5% | 0.4% | 0.3% |
| Total Unknown | Less than | Less than | Less than |
| Impurities | 1% | 0.75% | 0.5% |

Suitable packages or containers can be used to hold and dispense pharmaceutical formulations of the present disclosure for oral administration. In one embodiment, the package comprises a labeled blister package, ampules, dial dispenser package, or bottle. The bottle can be an amber glass bottle as previously described. The packages can be a single dose package or can contain multiple doses of the pharmaceutical formulation. The kits of the disclosure can also comprise a means for containing any type of packaging that houses the unit dosage forms, for example bottles or vials, which can (for example) be held in close confinement for commercial sale such as, e.g., injection or blow-molded plastic containers into which the bottles or vials are retained.

In an embodiment, the present disclosure comprises methods for manufacturing oral pharmaceutical formulations or methods for stabilizing oral pharmaceutical formulations comprising: adding one or more solubilizers to a clean manufacturing tank and purging the one or more solubilizers with a sparging agent, for example nitrogen, adding one or more dispersants to the manufacturing tank and rinsing the one or more dispersants using the one or more solubilizers. The rinsate can be transferred to the manufacturing tank and mixed with continuous purging and/or sparging with an inert gas (e.g., nitrogen). The immunosuppressive agent (e.g., Tacrolimus) can be screened through a mesh screen, weighed, added to the manufacturing tank, and mixed with the dispersants and solubilizers with continuous inert gas (e.g., nitrogen) purging to achieve a clear solution. The resulting solution can be transferred to a holding tank through a mesh screen and mixed with continuous inert gas (e.g., nitrogen) purging and/or sparging. The solution can be filled in de-ionized bottles, preferably amber glass bottles using flexible tubing with continuous nitrogen purging and sparging, and can be sealed with a cap.

In another embodiment, the present disclosure comprises methods for manufacturing oral liquid dosage formulations, preferably a non-aqueous solutions as described in paragraphs [0033]-[0053] above, comprising: adding one or more pharmaceutically acceptable solubilizers to a clean manufacturing tank and purging and/or sparging the one or more inert gases, for example nitrogen, adding the immunosuppressive agent such as Tacrolimus and any additional pharmaceutically acceptable excipients such as flavoring agents, sweeteners, preservatives, buffering agents, pH adjusting agents, dispersing agents, carriers or combinations thereof to the manufacturing tank with continuous purging and/or sparging with an inert gas (e.g., nitrogen). Additional solubilizers, liquid carriers or physiologically acceptable solvents may be added to obtain the desired final volume and the oral liquid dosage formulation, preferably a solution, can be filled into single or multiple dose container such as a de-ionized amber glass bottles with continuous nitrogen purging and/or sparging and sealed with a screw cap.

In certain embodiments the single or multiple dose amber glass bottles with the oral liquid dosage formulation, preferably the solution described above in paragraphs [0033]-[0053] are “ready to use”. Specifically the patient or care provider will measure the appropriate volume of the oral liquid into a dosing device such as a cup, spoon or syringe without further manipulation of the oral liquid dosage formulation such as suspending, diluting or combining with additional components. Once the appropriate amount volume of the oral liquid dosage formulation is in the dosing device, the patient or care provider may administered the oral liquid dosage formulation. In the case of a single dose bottle, the patient or care provider will not be required to measure the appropriate volume into a dosing device but may orally administer the oral liquid dosage formulation directly from the single dose bottle to the patient. In certain embodiments the oral liquid dosage form will not require any shaking, mixing or stirring prior to use or administration.

Further included herein are embodiments comprising methods of manufacturing or stabilizing the pharmaceutical formulation embodiments described herein. Also included are methods for treating an organ or stem cell transplant recipient, for preventing or lowering the risk of organ rejection, or for treating a T-cell mediated disease, comprising administering to a subject in need a pharmaceutical formulation according to the present disclosure. Also included are methods for inhibiting the effects of interleukin-2 (lowering the risk of organ rejection in a transplant patient/subject) comprising administering to the subject in need a pharmaceutical formulation according to the present disclosure.

Further included herein are embodiments comprising a dosage form or a kit comprising one or more dosage forms and instructions for administering the dosage forms to a subject in need, wherein the dosage forms comprise a pharmaceutical formulation according to the present disclosure, for treating an organ or stem cell transplant recipient or a T-cell mediated disease.

Even further included herein are therapeutic methods comprising administering to a subject in need of an oral pharmaceutical formulation according to the present disclosure, wherein administration of the formulation achieves a reduction in adverse events as compared to other formulations. In one embodiment, the adverse events being reduced can comprise cardiac damage, hypertension, blurred vision, liver problems, tacrolimus nephrotoxicity, hyperkalemia, loss of appetite, insomnia, seizures, and catatonia (among other things). In one embodiment, the reduction in adverse events comprises a reduction in the duration of a subject's pain, discomfort, inflammation and/or weakness (among other things). Even further included are methods for improving patient/subject compliance comprising administering to said patient/subject in need an oral pharmaceutical formulation according to the present disclosure.

Further included herein are pharmaceutical formulations according to the present disclosure, for inhibiting calcineurin. In one embodiment, the pharmaceutical formulation for inhibiting calcineurin comprises Tacrolimus. Further included herein are pharmaceutical formulations according to the present disclosure, for targeting FKBP12. In one embodiment, the pharmaceutical formulation for targeting FKBP12 comprises Tacrolimus.

Further included herein are therapeutic methods of inhibiting calcineurin. In one embodiment, the therapeutic methods of inhibiting calcineurin comprise administering to a subject in need a pharmaceutical formulation according to the present disclosure. Further included herein are therapeutic methods of targeting FKBP12. In one embodiment, the therapeutic methods of targeting FKBP12 comprise administering to a subject in need a pharmaceutical formulation according to the present disclosure. Further included herein are methods of increasing the bioavailability of an immunosuppressive agent in a patient. In one embodiment, the immunosuppressive agent is Tacrolimus.

### EXAMPLES

Below are examples of formulations of Tacrolimus oral solutions according to embodiments of the present disclosure. Comparative assessments of the bioavailability and pharmacokinetic parameters of Tacrolimus in solution dosage forms according to the present disclosure and that of capsule dosage forms can be conducted.

### Example 1

### Formulation Composition of Tacrolimus Oral Solution, 0.5 mg/mL & 1.0 mg/mL

TABLE 1

| | 0.5 mg/mL | 1.0 mg/mL | |
|---|---|---|---|
| | strength | strength | |
| Ingredients | % w/v | % w/v | Function |
| Tacrolimus | 0.05 | 0.1 | Active |
| Polysorbate 80, NF | 0.8 | 0.8 | Dispersant |
| Phosal 50 PG² | 99.65 | 99.60 | Solubilizer |
| Nitrogen, NF | QS | QS | Sparging Agent |

¹ Density of the product is approximately 1.005 g/mL.
²For composition of Phosal 50 PG, please see Table 6 below.
QS = Quantity Sufficient.

The compositions of Example 1 were prepared under yellow light throughout complete manufacturing according to the following procedure.
1. The Phosal 50 PG was added to a clean manufacturing tank and stirred using a propeller mixer.
2. The Phosal 50 PG was sparged for at least 10 minutes with Nitrogen NF supplied from a Nitrogen Cylinder with the tip of a nitrogen supply tube dipped into the Phosal 50 PG.
3. Polysorbate 80, NF was added to the manufacturing tank of Step #2 and the Polysorbate 80 container was rinsed with Phosal 50 PG and the rinsate transferred to the manufacturing tank. The resulting composition was mixed for 20 minutes with continuous Nitrogen sparging/purging.
4. Tacrolimus was screened through an 80 mesh stainless steel screen and added to the manufacturing tank of Step #3 and mixed for 10 minutes with continuous Nitrogen sparging/purging. After 10 minutes the propeller mixer was stopped, the lid of the manufacturing tank opened and the side walls scrapped with a stainless steel scrapper. After scrapping the lid was closed, the propeller mixer was turned “ON” and the composition was mixed further for 170 minutes with continuous Nitrogen sparging/purging to achieve a clear solution.
5. The solution from manufacturing tank of Step #4 was transferred to a holding tank through a 100 mesh stainless steel screen followed by industrial metal detector using a transfer pump using Platinum cured silicon tubing.
6. The propeller mixer of the holding tank was turned “ON” and solution was mixed for 20 minutes with continuous Nitrogen sparging/purging.
7. In-process samples were taken.
8. After the in-process samples were taken, the lid of the holding tank was tightly closed and the solution was continuously sparged/purged with Nitrogen until the bulk solution was filled and packaged.
9. Filling and Packaging Process:
9a. The holding tank was connected to the filling machine using flexible tubing (Platinum cured silicone) and the bulk solution was continuously sparged/purged with Nitrogen.
9b. The IPS Filling Machine was set to the required fill weight (2 oz per bottle).
9c. The Nitrogen supply from the cylinder should be set to also provide Nitrogen for one of the filling Nozzle of the Filling Machine.
9d. Amber glass bottles that had been de-ionized with air were filled the set amount of solution, approximately 2 oz. per bottle (Target Fill Weight).
9e. Once the bottle was filled to the Target Fill Weight it tightly the cap, using a Kaps All Capper.

### Example 2

### Formulation Composition of Tacrolimus Oral Solution, 0.5 mg/mL & 1.0 mg/mL

TABLE 2

| | 0.5 mg/mL | 1.0 mg/mL | |
|---|---|---|---|
| | strength | strength | |
| Ingredients | % w/v | % w/v | Function |
| Tacrolimus | 0.05 | 0.1 | Active |
| Polysorbate 80, NF | 1.0 | 1.0 | Dispersant |
| Phosal 50 PG² | 99.45 | 99.40 | Solubilizer |
| Nitrogen, NF | QS | QS | Sparging Agent |

¹ Density of the product is approximately 1.005 g/mL.
²For composition of Phosal 50 PG, please see Table 6 below.
QS = Quantity Sufficient.

The compositions of Example 2 were manufactured and packaged similar to the procedure described in Example 1.

### Example 3

### Formulation Composition of Tacrolimus Oral Solution, 0.5 mg/mL & 1.0 mg/mL

TABLE 3

| | 0.5 mg/mL | 1.0 mg/mL | |
|---|---|---|---|
| | strength | strength | |
| Ingredients | % w/v | % w/v | Function |
| Tacrolimus | 0.05 | 0.1 | Active |
| Polysorbate 80, NF | 1.2 | 1.2 | Dispersant |
| Phosal 50 PG² | 99.25 | 99.20 | Solubilizer |
| Nitrogen, NF | QS | QS | Sparging Agent |

¹ Density of the product is approximately 1.005 g/mL.
²For composition of Phosal 50 PG, please see Table 6 below.
QS = Quantity Sufficient.

The compositions of Example 3 were manufactured and packaged similar to the procedure described in Example 1.

Tables 4 and 5 present stability characteristics of the compositions provided in Examples 1 & 2 above.

TABLE 4
Example 1
Tacrolimus Oral Solution, 1 mg/mL
(Related Compound and Assay Data)
Related Compound

| | Tacrolimus Oral Solution, |
|---|---|
| | 1 mg/mL (Example: 1) |
| | (2-8)° C./12 Months |
| Impurity Name | % Impurity |
| Tacrolimus 8-Epimer | 0.047 |
| Tacrolimus Related | 0.012 |
| Compound A | |
| Tacrolimus 8-Propyl analog | ND |
| Tacrolimus Diene | 0.013 |
| Unknown-1 | 0.008@ RRT 1.25 |
| % Total Impurity | 0.080 |

Assay

| | Example: 1 | Example: 1 | Example: 1 |
|---|---|---|---|
| | (1.0 mg/mL strength) | (1.0 mg/mL strength) | (1.0 mg/mL strength) |
| | (2-8)° C./4 Months | 25° C./60% RH/4 Months | (2-8)° C./12 Months |
| | (% Assay) | (% Assay) | (% Assay) |
| % Assay | 102.9 | 100.8 | 99.5 |

TABLE 5
Example 2
Tacrolimus Oral Solution, 1 mg/mL
(Related Compound and Assay Data)
Related Compound

| | (Example 2; | (Example 2; | (Example 2; |
|---|---|---|---|
| | 1 mg/2 mL Strength) | 1 mg/2 mL Strength) | 1 mg/2 mL Strength) |
| | Initial | (2-8)° C./3 Months | 25° C./60% RH/3 Months |
| Impurity Name | % Impurity | % Impurity | % Impurity |
| Tacrolimus 8- | 0.052 | 0.087 | 0.267 |
| Epimer | | | |
| Tacrolimus Related | ND | 0.018 | 0.018 |
| Compound A | | | |
| Tacrolimus 8- | ND | ND | ND |
| Propyl analog | | | |
| Tacrolimus Diene | — | 0.006 | 0.027 |
| Unknown-1 | ND@ RRT 0.93 | 0.011@ RRT 1.25 | 0.062@ RRT 1.25 |
| % Total Impurity | 0.052 | 0.122 | 0.374 |

Assay

| | (Example 2; | (Example 2; | (Example 2; |
|---|---|---|---|
| | 1 mg/2 mL Strength) | 1 mg/2 mL Strength) | 1 mg/2 mL Strength) |
| | Initial | (2-8)° C./3 Months | 25° C./60% RH/3 Months |
| | (% Assay) | (% Assay) | (% Assay) |
| % Assay | 101.7 | 100.5 | 99.5 |

TABLE 6
PHOSAL ® 50 PG

| Ingredient | Content |
|---|---|
| Phosphatidylcholine | ≥50% |
| Propylene Glycol | 25-50% |
| Sunflower Seed Oil Glyceride | 1-5% |
| Soy Acid | 1-5% |
| Alcohol | 1-5% |
| Ascorbyl Palmitate | 0.1-1% |
| Tocopherol | 0.1-1% |

### Example 4

The following table exemplifies an oral liquid Tacrolimus dosage form that was prepared by a procedure similar to that described in Example 1:

| Ingredients | % w/v |
|---|---|
| Tacrolimus | 0.1% |
| Propylene Glycol | 40.0% |
| Purified water | 20% |
| Cyclodextrin | 20.0% |
| Sodium Saccharin | 0.05% |
| Glycerin | 10.0% |
| Methylparaben | 0.1% |
| Propylparaben | 0.02% |
| Purified Water | QS to 100% |

The following Tables 8-9 show purity of a Tacrolimus oral solution of 1 mg/ml as a percent assay, based on the batch descriptions in Table 7.

TABLE 7
Batch Description

| Batch # | Batch Composition | Batch Size |
|---|---|---|
| Example 3 (RD6185-05) | Batch with Phosal 50 PG | 2.0 Liter |
| Example 4 (RD6185-07) | Batch with Cyclodextrin | 2.0 Liter |

TABLE 8:
Assay for Example 3 (RD6185-05)[Batch with Phosal 50 PG]

| Batch # | % Assay |
|---|---|
| API_Lot # DMP (079)-38 | 97.1% |
| RD6185-05_CRT (3 Month) | 92.5% |
| RD6185-05_(2-8 C.) (3 Month) | 96.8% |

TABLE 9
Assay for Example 4 (RD6185-07)[Batch with Cyclodextrin]

| Batch # | % Assay |
|---|---|
| API_Lot # DMP (079)-38 | 97.1% |
| RD6185-07_CRT (Initial) | 89.9% |
| RD6185-07_(2-8 C.) (Initial) | 89.9% |

Tables 8-9 show the results of purity assays comparing the active pharmaceutical ingredient (API) with the batch compositions shown in Table 7 at room temperature (CRT) and refrigeration conditions (2-8° C.).

### Example 5

A Tacrolimus oral solution with the following composition was prepared:

| Ingredients | % w/v |
|---|---|
| Tacrolimus | 0.1% |
| Propylene Glycol | 30.0% |
| Saccharin Sodium | 0.2% |
| UB 1456 Orange Extract | 0.2% |
| Glycerin | QS to 100% |

The composition of Example 5 was prepared under yellow light throughout complete manufacturing according to the following procedure.
1. The Propylene Glycol was added to a clean manufacturing tank and stirred using a propeller mixer.
2. The Propylene Glycol was sparged for at least 10 minutes with Nitrogen NF supplied from a Nitrogen Cylinder with the tip of a nitrogen supply tube dipped into the Propylene Glycol.
3. The Tacrolimus was added to the manufacturing tank of Step #2 and the resulting composition was mixed for 15 minutes with continuous Nitrogen sparging/purging.
4. The Saccharin Sodium was added to the manufacturing tank of Step #3 and the resulting composition was mixed for 40 minutes with continuous Nitrogen sparging/purging.
5. The UB 1456 Orange Extract was added to the manufacturing tank of Step #4 and the resulting composition was mixed for 5 minutes with continuous Nitrogen sparging/purging
6. The solution from manufacturing tank of Step #5 was transferred to a holding tank through a 100 mesh stainless steel screen followed by industrial metal detector using a transfer pump using Platinum cured silicon tubing.
7. The propeller mixer of the holding tank was turned “ON” and solution was mixed for 20 minutes with continuous Nitrogen sparging/purging.
8. In-process samples were taken.
9. After the in-process samples were taken, the lid of the holding tank was tightly closed and the solution was continuously sparged/purged with Nitrogen until the bulk solution was filled and packaged.
10. Filling and Packaging Process:
10a. The holding tank was connected to the filling machine using flexible tubing (Platinum cured silicone) and the bulk solution was continuously sparged/purged with Nitrogen.
10b. The IPS Filling Machine was set to the required fill weight (2 oz per bottle).
10c. The Nitrogen supply from cylinder should be set to also provide Nitrogen for one of the filling Nozzle of the Filling Machine.
10d. Amber glass bottles that had been de-ionized with air were filled the set amount of solution, approximately 2 oz. per bottle (Target Fill Weight).
10e. Once the bottle was filled to the Target Fill Weight it tightly the cap, using a Kaps All Capper.

The composition of Example 5, packaged in 60 mL amber colored glass bottles with a tightly sealed child resistant closure, was placed on stability and showed the following stability profile:

### Related Compound:

| | Tacrolimus | Tacrolimus | Any | | | |
|---|---|---|---|---|---|---|
| | 8-Epimer | Diene | Unknown Imp | | | |
| Stability | Limit: | Limit: | Limit: | Total | Total | Total |
| Condition | 0.5% | 0.5% | 0.2% | Known Imp | Unknown Imp | Impurities |
| Initial | ND | ND | ND | ND | ND | ND |
| 2-8° C./3 m | ND | ND | ND | ND | ND | ND |
| 2-8° C./6 m | 0.038 | 0.062 | 0.033 | 0.1 | 0.03 | 0.13 |
| CRT/6 m | 1.032 | 1.08 | 0.264 | 2.112 | 0.5 | 2.612 |

*CRT = 25° C./60% relative humidity

### Assay Data:

| Stability Condition | Assay |
|---|---|
| Initial | 99.3 |
| 2-8° C./3 m | 102.7 |
| 2-8° C./6 m | 98.4 |
| CRT/6 m | 86.9 |

### Example 6

A Tacrolimus oral solution with the following composition was prepared:

| Ingredients | % w/v |
|---|---|
| Tacrolimus | 0.1% |
| Polyethylene Glycol 400 | 40.0% |
| Saccharin Sodium | 0.3% |
| UB 1456 Orange Extract | 0.2% |
| Propylene Glycol | QS to 100% |

The above solution was prepared and packaged according to the general procedure described in Example 5 with the following deviations:
1. Propylene glycol and polyethylene glycol 400 were initially added to manufacturing tank and mixed for 15 minutes with Nitrogen sparging/purging.
2. The Tacrolimus was added to the solution of step 1 and mixed for 30 minutes.
3. The saccharin sodium was added to the solution of step 2 and mixed for 40 minutes.
4. The UB 1456 Orange Extract was added to the solution of step 3 and mixed for 5 minutes.
5. Propylene Glycol was added to the solution of step 4 to make up the final volume and mixed for 10 minutes.

The invention illustratively described herein suitably may be practiced in the absence of any element or elements, limitation or limitations which is not specifically disclosed herein. Thus, for example, in each instance herein, any of the terms “comprising,” “consisting essentially of” and “consisting of” may be replaced with either of the other two terms. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. An oral liquid dosage formulation comprising:
(a) one or more immunosuppressive agents;
(b) one or more pharmaceutically acceptable solubilizers; and
(c) optionally one or more pharmaceutically acceptable excipients selected from the group consisting of flavoring agents, sweeteners, preservatives, buffering agents, pH adjusting agents, dispersants, carriers or combinations thereof
wherein the formulation is a non-aqueous solution.

2. The oral liquid dosage formulation of claim 1 wherein the one or more immunosuppressive agent is selected from the group consisting of cyclosporine, tacrolimus, sirolimus, everolimus and combinations thereof.

3. The oral liquid dosage formulation of claim 1 wherein the one or more pharmaceutically acceptable solubilizers are selected from the group consisting of selected from the group consisting of polyethylene glycols, propylene glycol, glycerin, triacetin, ethanol, polysorbates, oils and combinations thereof.

4. The oral liquid dosage formulation of claim 3 wherein the one or more pharmaceutically acceptable solubilizers are selected from the group consisting of selected from the group consisting of polyethylene glycols, propylene glycol, glycerin, triacetin, oils and combinations thereof.

5. The oral liquid dosage formulation of claim 1 wherein the one or more pharmaceutically acceptable solubilizers are liquid at room temperature.

6. The oral liquid dosage formulation of claim 1 wherein the formulation is substantially free of a surfactant, wetting agent or emulsifying agent.

7. The oral liquid dosage formulation of claim 1 wherein the formulation is substantially free of low molecular weight mono alcohols.

8. An oral liquid dosage formulation comprising:
(a) tacrolimus;
(b) one or more pharmaceutically acceptable solubilizers selected from the group consisting of selected from the group consisting of polyethylene glycols, propylene glycol, glycerin, triacetin, ethanol, polysorbates, oils and combinations thereof; and
(c) optionally one or more pharmaceutically acceptable excipients selected from the group consisting of flavoring agents, sweeteners, preservatives, buffering agents, pH adjusting agents, or combinations thereof
wherein the formulation is a non-aqueous solution, free of a surfactant, wetting agent, emulsifying agent and low molecular weight mono alcohols.
